(19) United States
(12) Patent Application Publication
(10) Pub. No.: US 2024/0165010 A1
(43) Pub. Date: May 23, 2024

(54) FACIAL MASK OR PATCH COMPOSITION USING OPTICAL STIMULATION AND COSMETIC

(71) Applicant: S.THEPHARM CO., LTD., Gyeonggi-do (KR)

(72) Inventors: Kyung Min Choi, Seoul (KR); Hyeon Shin Lee, Seoul (KR)

(21) Appl. No.: 17/641,839

(22) PCT Filed: May 14, 2020
(86) PCT No.: PCT/KR2020/006331

§ 371 Date: May 5, 2022

(30) Foreign Application Priority Data
Sep. 10, 2019 (KR) 1020190111881

(51) Int. Cl. A61K 8/73; A61K 8/02; A61Q 19/00

(52) U.S. Cl. CPC A61K 8/735; A61K 8/0212; A61Q 19/00; A61K 2800/81

(57) ABSTRACT

The present application relates to a facial mask composition using optical stimulation and a cosmetic product comprising same, and more specifically, to a facial mask cured by irradiating ultraviolet rays or visible light on a facial mask composition coated on the skin of a user. Accordingly, a facial mask or patch not requiring a facial mask or patch sheet may be provided, and a facial mask or patch having excellent applicability, adhesion properties and elasticity may be provided.

### TECHNICAL FIELD

The present application relates to a composition for a facial mask using optical initiation and a cosmetic product including the same, and more particularly, to a facial mask in which a composition for a facial mask applied on a user's skin is cured by irradiating UV light or visible light.

### TECHNICAL FIELD

Hydrogels, also called hydrated gels, are materials that have a network structure in which water-soluble polymers form a three-dimensional crosslink by a physical (hydrogen bond, Van der Waals force, hydrophobic interaction, or crystals of a polymer) or chemical (covalent bond) bond, and can contain a considerable amount of water without being dissolved in an aqueous environment. Since hydrogels are made from various water-soluble polymers, they have several chemical compositions and properties. In addition, these materials may be easy to process and modified into various forms according to an application. As can be seen from the successful application in the peritoneum and various parts of the body, hydrogels have high biocompatibility due to a high water content and physicochemical similarity with the extracellular matrix. Due to these characteristics, hydrogels are attracting attention as one of the most attractive materials for medical, pharmacological and cosmetic applications.

Korean Unexamined Patent Application Publication No. 10-2016-0055542 discloses a hydrogel facial pack manufactured by adding an essence to a formed body formed of a stand-alone hydrogel facial pack composition, thereby improving strength and adding various types of active ingredients which are sensitive to temperature. However, to form a hydrogel facial pack, since mixing, heating, cooling, drying and cutting are required, there was a disadvantage of the facial mask having a complicated production process.

However, there was no research on a method of simply forming a facial mask or patch by irradiating light at the time of use after being provided in a lotion type as a cosmetic product formulation.

### RELATED ART DOCUMENT

### Patent Document

(Patent Document 1) Korean Unexamined Patent Application Publication No. 10-2018-0102735 (published Sep. 18, 2018)

### DISCLOSURE

### Technical Problem

According to one embodiment of the present application, the present application is directed to providing various types of facial masks or patches by simple light irradiation to a cosmetic product applied to the skin at the time of use.

### Technical Solution

One aspect of the present application provides an optical stimulation-curable composition which includes poly(ethylene glycol) diacrylate (PEGDA), a photoinitiator, sodium hyaluronate, polyurethane and deionized water.

In one embodiment, the proportions of the components in the composition are 1 to 5 parts by weight of the photoinitiator, 5 to 10 parts by weight of sodium hyaluronate, 10 to 120 parts by weight of polyurethane, and 500 to 1000 parts by weight of deionized water, based on 100 parts by weight of PEGDA.

In another embodiment, the proportions of the components in the composition are 2.5 to 5 parts by weight of 2-hydroxy-4′-(2-hydroxyethoxy-2-methylpropiophenone), to 10 parts by weight of sodium hyaluronate, 11 to 56 parts by weight of polyurethane, and 500 to 1000 parts by weight of deionized water, based on 100 parts by weight of PEGDA.

In still another embodiment, the proportions of the components in the composition are 1.3 to 2.5 parts by weight of phenyl bis(2,4,6-trimethylbenzoyl)phosphine oxide, 5 to 10 parts by weight of sodium hyaluronate, 27 to 67 parts by weight of polyurethane, and 500 to 1000 parts by weight of deionized water, based on 100 parts by weight of PEGDA.

Another aspect of the present application provides a cosmetic product including the above-described composition, and the cosmetic product is a lotion-type formulation, which is cured by optical stimulation.

In one embodiment, light for the optical stimulation is UV light or visible light.

Still another aspect of the present application provides a method of preparing an optical stimulation-curable composition, which includes: preparing PEGDA; preparing a first mixture by mixing 500 to 1000 parts by weight of deionized water, based on 100 parts by weight of PEGDA, with the prepared PEGDA; and preparing a second mixture by mixing 1 to 5 parts by weight of a photoinitiator, 5 to 10 parts by weight of sodium hyaluronate and 10 to 120 parts by weight of polyurethane, based on 100 parts by weight of PEGDA, with the first mixture.

Yet another aspect of the present application provides a method of forming a facial mask or patch, which includes applying the above-described lotion-type cosmetic product to one area of a user's skin; and curing the applied product by irradiating light to a part of the area of skin to which the cosmetic product is applied.

### Advantageous Effects

According to one embodiment of the present application, a composition for a facial mask or patch can be provided.

According to one embodiment of the present application, various types of facial masks or patches by simply irradiating UV light or visible light on a cosmetic product applied to the skin at the time of use can be provided.

According to one embodiment of the present application, a facial mask or patch that does not need a sheet for a facial mask or patch can be provided.

According to one embodiment of the present application, a facial mask or patch having excellent applicability can be provided.

According to one embodiment of the present application, a facial mask or patch having excellent adhesion can be provided.

According to one embodiment of the present application, a facial mask or patch having excellent elasticity can be provided.

According to one embodiment of the present application, a facial mask or patch having no yellowing/discoloration can be provided.

### DESCRIPTION OF DRAWINGS

FIG. 1 is a flowchart illustrating a method of preparing an optical stimulation-curable composition according to one embodiment of the present application.

FIG. 2 is a flowchart illustrating a method of forming a facial mask or patch according to one embodiment of the present application.

FIG. 3 is an image illustrating a result of an adhesive test for a composition for a facial mask or patch according to one embodiment of the present application.

FIG. 4 is an image illustrating a result of an adhesive test for a composition for a facial mask or patch according to another embodiment of the present application.

FIG. 5 is an image illustrating a result of an adhesive test for a composition for a facial mask or patch according to still another embodiment of the present application.

FIG. 6 is an image illustrating a result of an adhesive test for a composition for a facial mask or patch according to yet another embodiment of the present application.

FIG. 7 is an image illustrating a result of an adhesive test for a composition for a facial mask or patch according to yet another embodiment of the present application.

### MODES OF THE INVENTION

The terms used herein are used only to describe specific examples, not to limit the present invention. Singular expressions include plural expressions unless the context clearly indicates otherwise. The terms “include” and “have” used herein designate the presence of characteristics or components described in the specification, and it does not mean that the possibility of the presence or addition of one or more other characteristics or components is excluded in advance.

All terms including technical and scientific terms have the same meaning that is generally understood by those skilled in the art unless defined otherwise. General terms, such as terms defined in dictionaries, should be interpreted with meanings according to the context of related art, and should not be interpreted with ideal or excessively formal meanings unless clearly defined herein.

Hereinafter, with reference to the accompanying drawings, an optical stimulation-curable composition, a cosmetic product, a method of preparing an optical stimulation-curable composition and a method of manufacturing a facial mask or patch according to the present invention will be described in detail. However, the accompanying drawings are exemplary, and the scope of an optical stimulation-curable composition, a cosmetic product, a method of preparing an optical stimulation-curable composition and a method of manufacturing a facial mask or patch according to the present invention are not limited by the accompanying drawings.

One aspect of the present application provides an optical stimulation-curable composition.

The optical stimulation-curable composition may include poly(ethylene glycol) diacrylate (PEGDA), a photoinitiator, sodium hyaluronate, polyurethane, and deionized water.

The optical stimulation-curable composition includes PEGDA. Poly(ethylene glycol) (PEG) is a type of polymer forming a network structure, and thus acts as a support that forms a hydrogel film by forming a network structure. In the case of PEGDA, when two acrylates respectively attached to both ends of PEG are used along with a photocuring accelerator, they are bonded to each other by light irradiation, thereby forming a network structure, and thus the PEGDA may be used as a support for a facial mask or patch.

In addition, the optical stimulation-curable composition includes a photoinitiator. The photoinitiator is included to establish a crosslinked structure between molecules. Light is a collection of particles called photons, but each photon has the properties of an electromagnetic wave. When a photon collides with a molecule that constitutes matter, the molecule receives the energy of the photon and enters a high energy state. The state of the molecule having a higher energy due to the photon collision is referred to as an excited state, and after collision, the molecule consumes the given energy (ΔE) and return to a ground state. According to this, the crosslinked structure between molecules is established by a photoreaction. In addition, a resin component is cured by the establishment (polymerization) of the crosslinked structure.

In the present application, the photoinitiator is not particularly limited as long as it is a material that has the above-described properties and is capable of crosslinking PEGDA. However, as an example, 2-hydroxyl-4′-(2-hydroxyethoxyl-2-methylpropiophenone) or phenylbis(2,4,6-trimethylbenzoyl)phosphine oxide may be used.

These may be materials under the trade names IRGACURE® 2959 and IRGACURE® 819, respectively.

As to be described below, they may response to UV light or visible light, respectively. Hereinafter, the present application will be mainly described with IRGACURE® 2959 and IRGACURE® 819, but the present application is not limited thereto.

In addition, the optical stimulation-curable composition includes sodium hyaluronate. The sodium hyaluronate is included as a humectant or thickening agent. Specifically, when applied to the skin, sodium hyaluronate may keep the skin moist with its strong moisturizing power that attracts water that is hundreds of times its own weight. It forms a thin transparent water-retaining film to serve to alleviate external stimuli. It also serves to repel bacteria, and thus prevent skin problems and help the immune system and healing system.

In addition, the optical stimulation-curable composition includes polyurethane. Polyurethane is generated by reacting polyols with isocyanate conjugates (R—(N═C═O)n≥2) under a condition of a hydroxyl catalyst (R′—(OH)N≥2) or UV activation. The properties of polyurethane depend on the types of reactants, such as an isocyanate and a polyol, and the long bonds contained in the polyol help the polymer to become more flexible and elastic, and an enormous amount of bonds help the polymer to be harder. When the polymer maintains an intermediate length of the two bonds, the polymer may be very elastic and maintain moderate rigidity.

In addition, the optical stimulation-curable composition includes deionized water. The deionized water serves as a solvent, and is harmless when applied to the skin.

Here, the proportions of the components in the composition are preferably 1 to parts by weight of a photoinitiator, 5 to 10 parts by weight of sodium hyaluronate, 10 to 120 parts by weight of polyurethane, and 500 to 1000 parts by weight of deionized water, based on 100 parts by weight of PEGDA.

As to be described below, depending on the type of photoinitiator, the type of irradiated light is different, and the proportions of other elements are different.

Here, when the photoinitiator is 2-hydroxy-4′-(2-hydroxyethoxy-2-methylpropiophenone), the proportions of the components in the composition are preferably 2.5 to 5 parts by weight of 2-hydroxy-4′-(2-hydroxyethoxy-2-methylpropiophenone), 5 to 10 parts by weight of sodium hyaluronate, 11 to 56 parts by weight of polyurethane, and 500 to 1000 parts by weight of deionized water, based on 100 parts by weight of PEGDA.

When the content of 2-hydroxy-4′-(2-hydroxyethoxy-2-methylpropiophenone) is less than 2.5 parts by weight, the crosslinking reaction intended by the present application does not occur, and when the content is more than 5 parts by weight, the elasticity of a facial mask deteriorates.

In addition, when the content of sodium hyaluronate is less than 5 parts by weight, the viscosity decreases, and when the content is more than 10 parts by weight, the viscosity excessively increases.

In addition, when the content of polyurethane is less than 11 parts by weight, the elasticity of a facial mask decreases, and when the content is more than 56 parts by weight, yellowing occurs.

In addition, when the content of deionized water is less than 500 parts by weight, the viscosity is high, and when the content is more than 1000 parts by weight, the viscosity is low.

In addition, when the photoinitiator is phenyl bis(2,4,6-trimethylbenzoyl)phosphine oxide, the proportions of the components in the composition are preferably 1.3 to 2.5 parts by weight of phenyl bis(2,4,6-trimethylbenzoyl)phosphine oxide, 5 to 10 parts by weight of sodium hyaluronate, 27 to 67 parts by weight of polyurethane, and 500 to 1000 parts by weight of deionized water, based on 100 parts by weight of PEGDA.

When the content of phenyl bis(2,4,6-trimethylbenzoyl)phosphine oxide is less than 1.3 parts by weight, the crosslinking reaction intended by the present application does not occur, and when the content is more than 2.5 parts by weight, the elasticity of a facial mask deteriorates.

In addition, when the content of sodium hyaluronate is less than 5 parts by weight, the viscosity is low, and when the content is more than 10 parts by weight, the viscosity increases.

In addition, when the content of polyurethane is less than 27 parts by weight, the elasticity of a facial mask deteriorates, and when the content is more than 67 parts by weight, yellowing occurs.

In addition, when the content of deionized water is less than 500 parts by weight, the viscosity is high, and when the content is more than 1000 parts by weight, the viscosity is low.

The composition having the above-described component system may be cured by optical stimulation, and may be used for a facial mask or patch.

Another aspect of the present application provides a cosmetic product.

As a cosmetic product including the above-described composition, the cosmetic product may be a lotion-type formulation, which may be cured by optical stimulation.

The lotion-type cosmetic product may have a viscosity to be applied to a user's skin.

Particularly, when 2-hydroxy-4′-(2-hydroxyethoxy-2-methylpropiophenone) is used as a photoinitiator, the composition is cured by UV light, and the UV wavelength range is 340 to 390 nm.

In addition, when phenyl bis(2,4,6-trimethylbenzoyl)phosphine oxide is used as a photoinitiator, the composition is cured by visible light, and the visible light wavelength range is 380 to 430 nm.

Still another aspect of the present application provides a method of preparing an optical stimulation-curable composition.

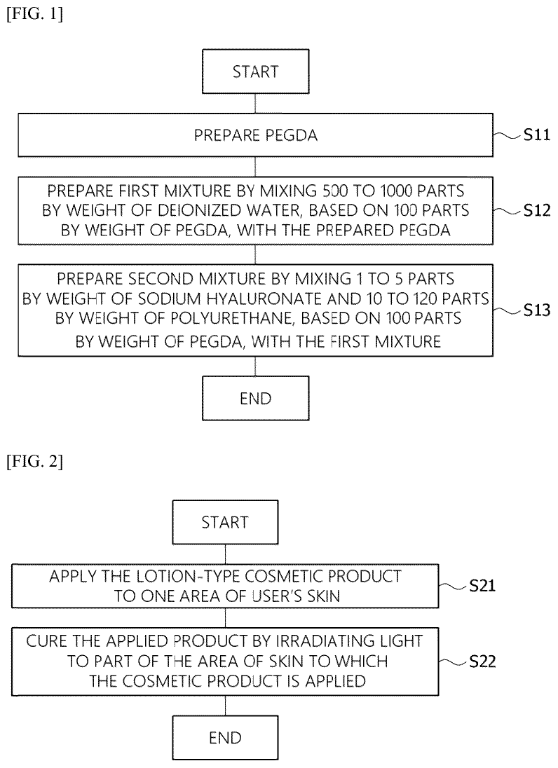

FIG. 1 is a flowchart illustrating a method of preparing an optical stimulation-curable composition according to one embodiment of the present application.

As shown in FIG. 1, PEGDA is prepared (S11).

In addition, a first mixture is prepared by mixing 500 to 1000 parts by weight of deionized water with the prepared PEGDA, based on 100 parts by weight of PEGDA (S12).

In addition, a second mixture is prepared by mixing 1 to 5 parts by weight of a photoinitiator, 5 to 10 parts by weight of sodium hyaluronate and 10 to 120 parts by weight of polyurethane with the first mixture, based on 100 parts by weight of PEGDA (S13).

As described above, when 2-hydroxy-4′-(2-hydroxyethoxy-2-methylpropiophenone) is used as a photoinitiator, the second mixture preferably includes 2.5 to 5 parts by weight of 2-hydroxy-4′-(2-hydroxyethoxy-2-methylpropiophenone), 5 to 10 parts by weight of sodium hyaluronate and 11 to 56 parts by weight of polyurethane, based on 100 parts by weight of PEGDA.

In addition, when 819 is used as a photoinitiator, the second mixture preferably includes 1.3 to 2.5 parts by weight of phenyl bis(2,4,6-trimethylbenzoyl)phosphine oxide, 5 to 10 parts by weight of sodium hyaluronate and 27 to 67 parts by weight of polyurethane, based on 100 parts by weight of PEGDA.

Here, each component and its proportion are replaced with the content related to the above-described composition, and in particular, additional description will be omitted.

Yet another aspect of the present application provides a method of forming a facial mask or patch.

FIG. 2 is a flowchart illustrating a method of forming a facial mask or patch according to one embodiment of the present application.

As shown in FIG. 2, a lotion-type cosmetic product is applied to one area of a user's skin (S21). In addition, the applied product is cured by irradiating light to a part of the area of skin to which the cosmetic product is applied (S22).

The cosmetic product includes PEGDA, and 2.5 to 5 parts by weight of 2-hydroxy-4′-(2-hydroxyethoxy-2-methylpropiophenone), 5 to 10 parts by weight of sodium hyaluronate, 11 to 56 parts by weight of polyurethane and 500 to 1000 parts by weight of deionized water, based on 100 parts by weight of PEGDA, and the light is preferably UV light.

In addition, the cosmetic product includes PEGDA, and 1.3 to 2.5 parts by weight of phenyl bis(2,4,6-trimethylbenzoyl)phosphine oxide, 5 to 10 parts by weight of sodium hyaluronate 27 to 67 parts by weight of polyurethane and 500 to 1000 parts by weight of deionized water, based on 100 parts by weight of PEGDA, and the light is preferably visible light.

Accordingly, the cosmetic product may be applied to the skin, particularly, the face, in the form desired by a user, and cured by simply irradiating UV light or visible light to be used like a facial mask or patch.

After a predetermined period of time, the cured facial mask or patch may be easily removed from the face according to the user's intention.

Hereinafter, the present application will be described in detail with reference to experimental examples.

### Experimental Example 1

According to the proportions of the components shown in Table 1 below, PEGDA and deionized water was put into a vial, and then 2-hydroxy-4′-(2-hydroxyethoxy-2-methylpropiophenone), sodium hyaluronate, and polyurethane were sequentially input. In addition, after mixing, the resulting mixture was poured into a petri dish, and irradiated with light with a wavelength of 340 to 390 nm for 30 seconds. For each example and comparative examples, applicability, adhesion (shape retention), elasticity, and yellowing were measured. The measurement results are shown in Table 1.

Specifically, applicability was confirmed by visually observing the degree of spreading of formulations when the formulations were applied in a circular shape with a diameter of approximately 3 cm. As the spread shape was close to a circle, it was denoted as excellent (⊚), and sequentially determined as º, Δ, and X, and the result is shown in Table 1.

In addition, the adhesion (shape retention) of a facial mask or patch was evaluated by repeating detachment and adhesion five times or more, and as the shape retention was excellent, it was denoted as ⊚. Sequentially, the shape retention was determined as º, Δ, and X, and the result is shown in Table 1.

In addition, when the facial mask or patch is detached, in an approximately 45-degree bending test, when there was no circular crack, it was denoted as ⊚. Sequentially, the elasticity was determined as º, Δ, and X, and the result is shown in Table 1.

In addition, the presence or absence of yellowing was evaluated by visually observing the surface of a facial mask or patch, and when there was no yellowing, it was denoted as ⊚. The elasticity was sequentially determined as º, Δ, and X, and the result is shown in Table 1.

However, adhesion that makes the form fixed as a facial mask or patch by light irradiation is the most important evaluation factor in the present application, and elasticity is an optional property in the present application.

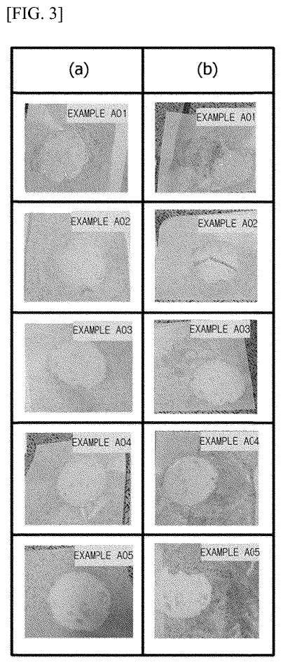

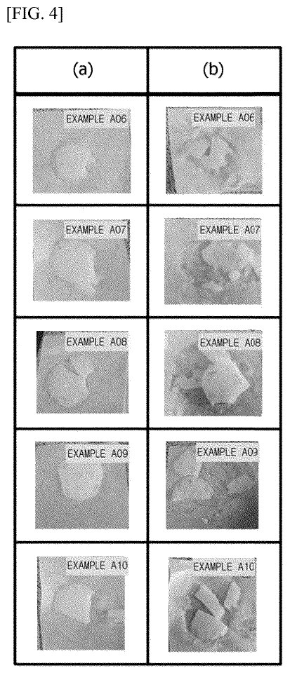

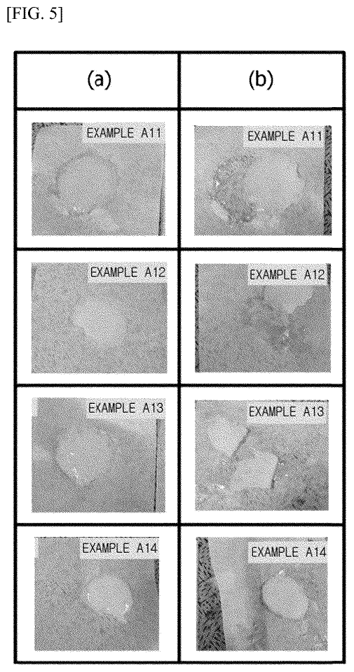

In addition, photographs were taken before and after adhesion tests for Examples A01 to A14, and are shown in FIGS. 3 to 5.

TABLE 1

| | | | | | | | | Adhesion/ | | |
|---|---|---|---|---|---|---|---|---|---|---|
| | PEGDA | HMM | HA | PU | DI | Irradiation | | Shape | | |
| Classification | (g) | (g) | (g) | (g) | (g) | Time | Applicability | retention | Elasticity | Yellowing |
| Comparative | 100 | 0 | 10 | 0 | 1000 | 30 sec | ◯ | X | X | X |
| Example A01 | | | | | | | | | | |
| Comparative | 100 | 5 | 10 | 0 | 1000 | 30 sec | ◯ | X | X | X |
| Example A02 | | | | | | | | | | |
| Example A01 | 100 | 5 | 10 | 11.1 | 1000 | 30 sec | ◯ | Δ | Δ | Δ |
| Example A02 | 100 | 5 | 10 | 22.3 | 1000 | 30 sec | ◯ | Δ | Δ | Δ |
| Example A03 | 100 | 5 | 10 | 33.5 | 1000 | 30 sec | ◯ | ◯ | ◯ | Δ |
| Example A04 | 100 | 5 | 10 | 44.6 | 1000 | 30 sec | ◯ | ⊚ | ⊚ | ◯ |
| Example A05 | 100 | 5 | 10 | 55.8 | 1000 | 30 sec | ◯ | ◯ | ◯ | ◯ |
| Comparative | 100 | 0 | 6.67 | 0 | 667 | 30 sec | ◯ | X | X | X |
| Example A03 | | | | | | | | | | |
| Comparative | 100 | 3.3 | 6.67 | 0 | 667 | 30 sec | ◯ | X | X | X |
| Example A04 | | | | | | | | | | |
| Example A06 | 100 | 3.3 | 6.67 | 7.8 | 667 | 30 sec | ◯ | Δ | Δ | Δ |
| Example A07 | 100 | 3.3 | 6.67 | 15.5 | 667 | 30 sec | ◯ | Δ | Δ | Δ |
| Example A08 | 100 | 3.3 | 6.67 | 23.3 | 667 | 30 sec | ◯ | ◯ | ⊚ | Δ |
| Example A09 | 100 | 3.3 | 6.67 | 31.0 | 667 | 30 sec | ◯ | ◯ | ⊚ | ◯ |
| Example A10 | 100 | 3.3 | 6.67 | 38.9 | 667 | 30 sec | ◯ | ◯ | ◯ | ◯ |
| Comparative | 100 | 0 | 5 | 0 | 500 | 30 sec | ◯ | X | X | X |
| Example A05 | | | | | | | | | | |
| Comparative | 100 | 2.5 | 5 | 0 | 500 | 30 sec | ◯ | X | X | X |
| Example A06 | | | | | | | | | | |
| Example A11 | 100 | 2.5 | 5 | 12.2 | 500 | 30 sec | ◯ | ◯ | X | ◯ |
| Example A12 | 100 | 2.5 | 5 | 18.3 | 500 | 30 sec | ◯ | ◯ | X | ◯ |
| Example A13 | 100 | 2.5 | 5 | 24.3 | 500 | 30 sec | ◯ | ◯ | X | ◯ |
| Example A14 | 100 | 2.5 | 5 | 30.4 | 500 | 30 sec | ◯ | ◯ | X | ◯ |

(HMM: 2-hydroxy-4′-(2-hydroxyethoxy-2-methylpropiophenone))

As shown in Table 1, Examples A01 to A16 satisfying the component system of the present application had satisfactory adhesion.

On the other hand, in Comparative Examples A01 and A02 that do not include polyurethane or a photoinitiator, curing did not occur, and even though light was additionally irradiated for up to 10 minutes or more, curing did not occur at all.

In addition, even in Comparative Examples A03 to A06 not including polyurethane or a photoinitiator, no curing occurred even when the light irradiation time was extended.

### Experimental Example 2

According to the proportions of the components shown in Table 2 below, PEGDA and deionized water were put into a vial, and phenyl bis(2,4,6-trimethylbenzoyl)phosphine oxide, sodium hyaluronate, and polyurethane were sequentially input. In addition, after mixing, the resulting mixture was poured into a petri dish, and irradiated with light with a wavelength of 380 to 430 nm for 2 or 6 minutes. For each of the examples and comparative examples, applicability, adhesion (shape retention), elasticity, and yellowing were measured. The measurement results are shown in Table 2.

Methods of measuring applicability, adhesion (shape retention), elasticity and yellowing were performed by the same methods as described in Experimental Example 1.

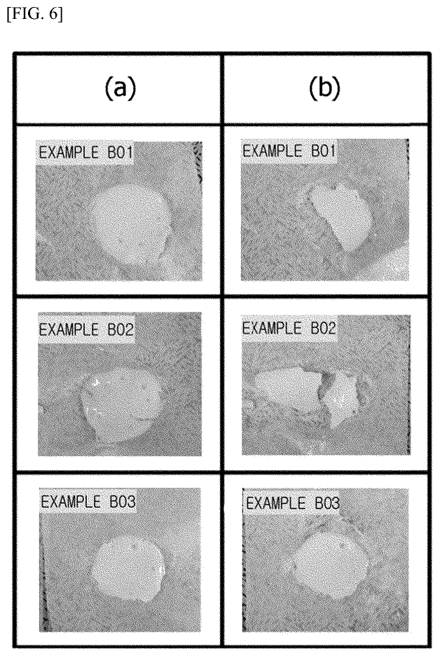

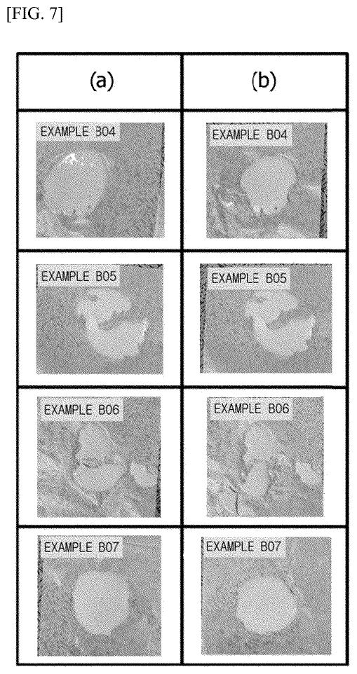

In addition, photographs were taken before and after adhesion tests for Examples B01 to B07 and are shown in FIGS. 6 and 7.

TABLE 2

| | | | | | | Irradiation | | Adhesion/ | | |
|---|---|---|---|---|---|---|---|---|---|---|
| Classification | PEGDA | 819 | HA | PU | DI | Time | Applicability | Shape retention | Elasticity | Yellowing |
| Comparative | 100 | 0 | 10 | 0 | 1000 | 2 min | ◯ | X | X | X |
| Example B01 | | | | | | | | | | |
| Comparative | 100 | 2.5 | 10 | 0 | 1000 | 2 min | ◯ | X | X | X |
| Example B02 | | | | | | | | | | |
| Example B01 | 100 | 2.5 | 10 | 55.6 | 1000 | 2 min | ◯ | Δ | ⊚ | X |
| Example B02 | 100 | 2.5 | 10 | 61.2 | 1000 | 2 min | ◯ | Δ | ⊚ | X |
| Example B03 | 100 | 2.5 | 10 | 66.8 | 1000 | 2 min | ◯ | ◯ | ⊚ | X |
| Comparative | 100 | 0 | 6.7 | 0 | 667 | 2 min | ◯ | X | X | X |
| Example B03 | | | | | | | | | | |
| Comparative | 100 | 1.6 | 6.7 | 0 | 667 | 2 min | ◯ | X | X | X |
| Example B04 | | | | | | | | | | |
| Example B04 | 100 | 1.6 | 6.7 | 34.9 | 667 | 2 min | ◯ | ◯ | ⊚ | X |
| Example B05 | 100 | 1.6 | 6.7 | 38.7 | 667 | 2 min | ◯ | ◯ | ◯ | X |
| Example B06 | 100 | 1.6 | 6.7 | 42.6 | 667 | 2 min | ◯ | Δ | ◯ | X |
| Example B07 | 100 | 1.6 | 6.7 | 46.5 | 667 | 2 min | ◯ | ◯ | ⊚ | X |
| Comparative | 100 | 0 | 5 | 0 | 500 | 2 min | ◯ | X | X | X |
| Example B05 | | | | | | | | | | |
| Comparative | 100 | 1.3 | 5 | 0 | 500 | 2 min | ◯ | X | X | X |
| Example B06 | | | | | | | | | | |
| Example B08 | 100 | 1.3 | 5 | 27.3 | 500 | 2 min | ◯ | Δ | X | X |
| Example B09 | 100 | 1.3 | 5 | 30.3 | 500 | 2 min | ◯ | Δ | X | X |
| Example B10 | 100 | 1.3 | 5 | 33.4 | 500 | 2 min | ◯ | Δ | X | X |
| Example B11 | 100 | 1.3 | 5 | 36.4 | 500 | 2 min | ◯ | Δ | X | X |

(IRGACURE ® 819: phenyl bis(2,4,6-trimethylbenzoyl)phosphine oxide)

As shown in Table 2, Examples B01 to B11 satisfying the component system of the present application had excellent adhesion.

On the other hand, even in Comparative Examples B01 to B06 that do not include polyurethane or a photoinitiator, curing did not occur even though light was additionally irradiated for up to 10 minutes or more.

In the above, exemplary embodiments of the present application have been described, but it will be understood by those of ordinary skill in the art that the present application may be changed and modified in various ways by modification and alteration without departing from the spirit of the present invention defined in the appended claims.

1. An optical stimulation-curable composition, comprising:
poly(ethylene glycol) diacrylate (PEGDA), a photoinitiator, sodium hyaluronate, polyurethane and deionized water.

2. The composition of claim 1, wherein the proportions of the components in the composition are 1 to 5 parts by weight of the photoinitiator, 5 to 10 parts by weight of sodium hyaluronate, 10 to 120 parts by weight of polyurethane, and 500 to 1000 parts by weight of deionized water, based on 100 parts by weight of PEGDA.

3. The composition of claim 1, wherein the proportions of the components in the composition are 2.5 to 5 parts by weight of 2-hydroxy-4′-(2-hydroxyethoxy-2-methylpropiophenone), 5 to 10 parts by weight of sodium hyaluronate, 11 to 56 parts by weight of polyurethane, and 500 to 1000 parts by weight of deionized water, based on 100 parts by weight of PEGDA.

4. The composition of claim 1, wherein the proportions of the components in the composition are 1.3 to 2.5 parts by weight of phenyl bis(2,4,6-trimethylbenzoyl)phosphine oxide, 5 to 10 parts by weight of sodium hyaluronate, 27 to 67 parts by weight of polyurethane, and 500 to 1000 parts by weight of deionized water, based on 100 parts by weight of PEGDA.

5. A cosmetic product comprising the composition of claim 1, which is a lotion-type formulation, which is cured by optical stimulation.

6. The cosmetic product of claim 5, wherein the optical stimulation is performed with UV light or visible light.

7. A method of preparing an optical stimulation-curable composition, comprising:
preparing PEGDA;
preparing a first mixture by mixing 500 to 1000 parts by weight of deionized water, based on 100 parts by weight of PEGDA, with the prepared PEGDA; and
preparing a second mixture by mixing 1 to 5 parts by weight of a photoinitiator, to 10 parts by weight of sodium hyaluronate and 10 to 120 parts by weight of polyurethane, based on 100 parts by weight of PEGDA, with the first mixture.

8. The method of claim 7, wherein the second mixture comprises 2.5 to 5 parts by weight of 2-hydroxy-4′-(2-hydroxyethoxy-2-methylpropiophenone), 5 to 10 parts by weight of sodium hyaluronate, 11 to 56 parts by weight of polyurethane, and 500 to 1000 parts by weight of deionized water, based on 100 parts by weight of PEGDA.

9. The method of claim 7, wherein the second mixture comprises 1.3 to 2.5 parts by weight of phenyl bis(2,4,6-trimethylbenzoyl)phosphine oxide, 5 to 10 parts by weight of sodium hyaluronate, and 27 to 67 parts by weight of polyurethane, based on 100 parts by weight of PEGDA.

10. A method of forming a facial mask or patch, comprising:
applying the lotion-type cosmetic product of claim 5 to one area of a user's skin; and
curing the applied product by irradiating light to a part of the area of skin to which the cosmetic product is applied.

11. The method of claim 10, wherein the cosmetic product comprises PEGDA, and 2.5 to 5 parts by weight of 2-hydroxy-4′-(2-hydroxyethoxy-2-methylpropiophenone), 5 to 10 parts by weight of sodium hyaluronate, 11 to 56 parts by weight of polyurethane and 500 to 1000 parts by weight of deionized water, based on 100 parts by weight of PEGDA, and
the light is UV light.

12. The method of claim 10, wherein the cosmetic product comprises PEGDA, and 1.3 to 2.5 parts by weight of phenyl bis(2,4,6-trimethylbenzoyl)phosphine oxide, 5 to 10 parts by weight of sodium hyaluronate 27 to 67 parts by weight of polyurethane and 500 to 1000 parts by weight of deionized water, based on 100 parts by weight of PEGDA, and
the light is visible light.
